(19) United States
(12) Patent Application Publication
(10) Pub. No.: US 2017/0000435 A1
(43) Pub. Date: Jan. 5, 2017

(54) BREAST IMAGING APPARATUS

(71) Applicant: CANON KABUSHIKI KAISHA, Tokyo (JP)

(72) Inventors: Tetsuo Shimada, Hachioji-shi (JP); Osamu Tsujii, Kawasaki-shi (JP); Hiroshi Komatsu, Yokohama-shi (JP); Sakiko Yamaguchi, Tokyo (JP); Nobuhiro Takeuchi, Yokohama-shi (JP); Takahiro Noguchi, Tokyo (JP); Hitomi Ogasawara, Kawasaki-shi (JP); Chifuyu Inagaki, Tokyo (JP); Yoshinori Hirano, Kashiwa-shi (JP)

(21) Appl. No.: 15/195,254

(22) Filed: Jun. 28, 2016

(30) Foreign Application Priority Data
Jun. 30, 2015 (JP) 2015-132186

(51) Int. Cl. A61B 6/00; A61B 6/04; A61B 6/03

(52) U.S. Cl. CPC A61B 6/4435; A61B 6/032; A61B 6/502; A61B 6/04; A61B 6/5211

(57) ABSTRACT

A breast imaging apparatus includes a gantry which includes a radiation generation unit configured to generate a radiation and a radiation detection unit configured to detect the radiation generated from the radiation generation unit, and a support leg portion configured to support the gantry on a floor. The radiation generation unit and the radiation detection unit are capable of rotating in a state where the radiation generation unit and the radiation detection unit face each other. The support leg portion has a recess portion to insert feet of a subject.

### BACKGROUND OF THE INVENTION

Field of the Invention

The present invention relates to a breast imaging apparatus that radiographs a breast of a subject.

Description of the Related Art

As a breast imaging apparatus, there is an apparatus that radiographs a breast using a radiation generation unit for generating a radiation and a radiation detection unit for detecting the radiation.

Some breast imaging apparatuses implement both mammogram imaging and computed tomographic (CT) imaging (refer to, for example, Japanese Patent Application Laid-Open No. 2013-538668). According to the breast imaging apparatus discussed in Japanese Patent Application Laid-Open No. 2013-538668, the breast of the subject is fixed between two plates in the CT imaging. Therefore, it is difficult to radiograph the breast in a state in which the breast is properly fixed.

### SUMMARY OF THE INVENTION

The present invention is directed to providing a breast imaging apparatus to perform CT imaging on the breast in a state to fix the breast properly. According to an aspect of the present invention, a breast imaging apparatus includes a gantry which includes a radiation generation unit configured to generate a radiation and a radiation detection unit configured to detect the radiation emitted from the radiation generation unit, and the radiation generation unit and the radiation detection unit are capable of rotating in a state where the radiation generation unit and the radiation detection unit face each other. The breast imaging apparatus includes a support leg portion configured to support the gantry with respect to a floor, wherein the support leg portion has a recess portion to allow a part of a foot (feet) of a subject to enter therein.

Further features of the present invention will become apparent from the following description of exemplary embodiments with reference to the attached drawings.

### BRIEF DESCRIPTION OF THE DRAWINGS

FIG. 1 is an external view of a breast imaging apparatus according to an exemplary embodiment of the present invention.

FIG. 2 is an external view of the breast imaging apparatus according to the exemplary embodiment when viewed from a computed tomographic (CT) imaging side.

FIG. 3 is a sectional view of the breast imaging apparatus.

FIG. 4 is a diagram illustrating a configuration of the breast imaging apparatus.

FIGS. 5A and 5B are diagrams each illustrating a support leg portion of the breast imaging apparatus.

FIG. 6 is a diagram illustrating a state of a subject during CT imaging.

FIG. 7 is a diagram illustrating a state of a subject during mammogram imaging.

### DESCRIPTION OF THE EMBODIMENTS

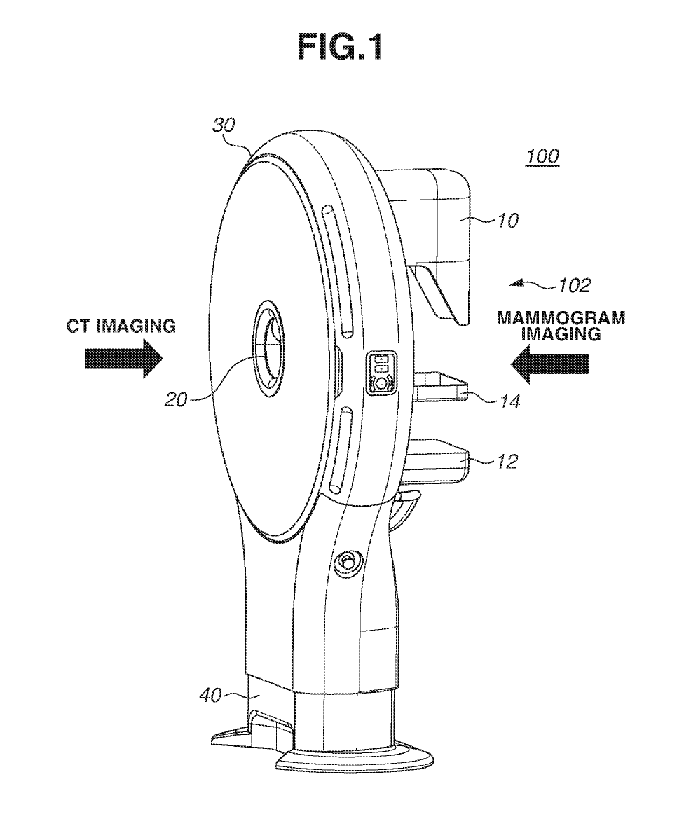

FIG. 1 is an external view of a breast imaging apparatus 100. The breast imaging apparatus 100 implements computer tomography (CT) imaging and mammogram imaging. When CT imaging and mammogram imaging are implemented, a subject is in a standing posture. The standing posture means that both feet of the subject are placed on the floor and the subject is standing on the floor. In other words, the breast imaging apparatus 100 is a breast imaging apparatus for the standing posture.

The breast imaging apparatus 100 includes a radiation generation unit 10 for generating radiation and a radiation detection unit 12 for detecting the radiation emitted from the radiation generation unit 10. The radiation generation unit 10 and the radiation detection unit 12 are capable of rotating in a state where the radiation generation unit 10 and the radiation detection unit 12 face each other. An imaging unit 102 includes mainly the radiation generation unit 10 and the radiation detection unit 12.

The breast imaging apparatus 100 performs a radiograph in a state where an imaging target portion (breast) of the subject on a first side of the breast imaging apparatus 100 (right side in FIG. 1) is pressed by a pressing plate 14. In the present exemplary embodiment, the imaging target portion (breast) is pressed between the pressing plate 14 and the radiation detection unit 12. Furthermore, the breast imaging apparatus 100 may include a grid (not illustrated) on the upper surface of the radiation detection unit 12, and a radiograph may be performed in a state where the imaging target portion (breast) is pressed between the pressing plate 14 and the grid. In other words, the breast imaging apparatus 100 has a first imaging means used in a mammogram imaging mode. Furthermore, the breast imaging apparatus 100 performs the radiograph while rotating the radiation generation unit 10 and the radiation detection unit 12 in a state where an imaging target portion (breast) of the subject is inserted, from a second side opposite to the first side, between the radiation generation unit 10 and the radiation detection unit 12. In other words, the breast imaging apparatus 100 has a second imaging means used in a CT imaging mode.

The breast imaging apparatus 100 includes a gantry 30 for rotatably supporting the radiation generation unit 10 and the radiation detection unit 12, and a support leg portion 40 for supporting the gantry 30 with respect to the floor. In other words, the gantry 30 supports the imaging unit 102 rotatably.

In mammogram imaging, the breast imaging apparatus 100 performs the radiograph in the state where an imaging target portion (breast) of the subject on the first side (right side in FIG. 1) of the breast imaging apparatus 100 is pressed between the pressing plate 14 and the radiation detection unit 12. The pressing plate 14 is made of a transparent material. The pressing plate 14 allows radiation to penetrate there through. Concretely, by moving the pressing plate 14 up and down, the breast of the subject is able to be pressed between the pressing plate 14 and the radiation detection unit 12. The radiation generation unit 10 generates the radiation in the state where the breast of the subject is pressed between the pressing plate 14 and the radiation detection unit 12. The radiation detection unit 12 detects the radiation passing through the breast of the subject. Therefore, it is possible to capture a radiographic image of the breast of the subject. The breast imaging apparatus 100 can generate a mammogram image based on the radiographed radiation data.

In CT imaging, an imaging target portion (breast) of the subject is inserted between the radiation generation unit 10 and the radiation detection unit 12 from the second side (left side in FIG. 1) opposite to the first side of the breast imaging apparatus 100. In such a state, the breast imaging apparatus 100 performs the radiograph while rotating the radiation generation unit 10 and the radiation detection unit 12 by a rotation frame 38. Concretely, an opening 20 for the subject to insert the breast is provided to the gantry 30 of the breast imaging apparatus 100. The breast imaging apparatus 100 performs the radiograph while rotating the radiation generation unit 10 and the radiation detection unit 12 by the rotation frame 38 in the state where the breast of the subject is inserted into the opening 20. While the breast imaging apparatus 100 is rotating the radiation generation unit 10 and the radiation detection unit 12 with the rotation frame 38, the radiation generation unit 10 generates the radiation. The radiation detection unit 12 detects the radiation passing through the breast of the subject. Therefore, it is possible to capture a radiographic image of the breast of the subject. The breast imaging apparatus 100 reconstructs the radiographed radiation data and generates a CT image.

The first side of the breast imaging apparatus 100 is a mammogram imaging side. The second side of the breast imaging apparatus 100 is a CT imaging side. A line which links the second side (the CT imaging side) to the first side (the mammogram imaging side) horizontally is substantially parallel to the rotation axis of the rotation frame 38. Moreover, the line which links the second side (the CT imaging side) to the first side (the mammogram imaging side) horizontally is orthogonal to a plane of the gantry 30 having a nearly planar shape or a plane of a front cover 26.

The first side (the mammogram imaging side) and the second side (the CT imaging side) of the breast imaging apparatus 100 are regions divided by the gantry 30 having the nearly planar shape, the front cover 26, and the imaging unit 102 of the breast imaging apparatus 100.

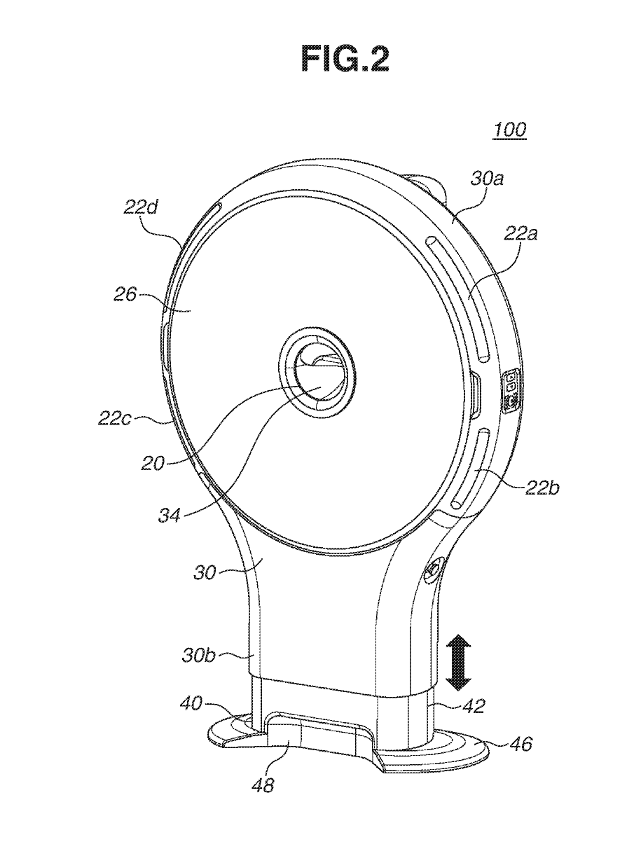

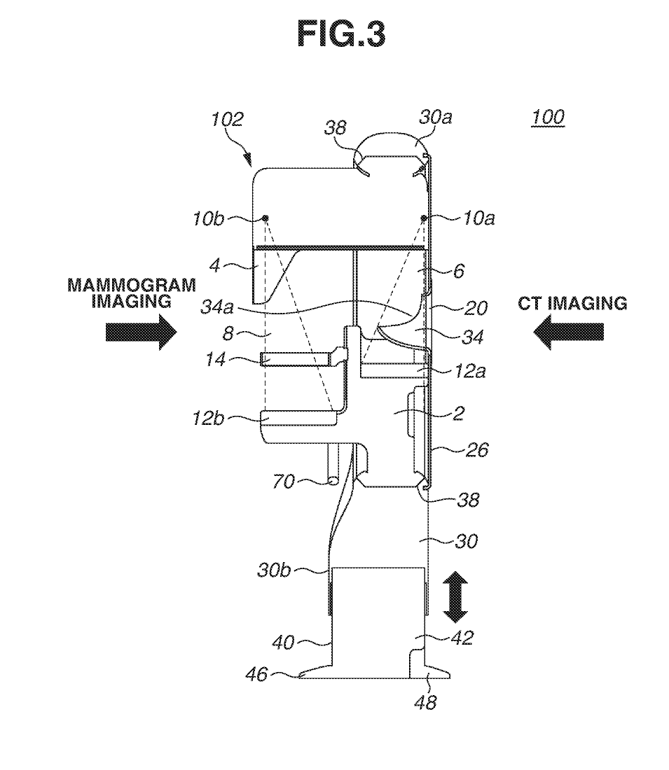

Here, the breast imaging apparatus 100 is described in detail with reference to FIG. 2 and FIG. 3. FIG. 2 is an external view of the breast imaging apparatus 100 when viewed from the CT imaging side. FIG. 3 is a sectional view of the breast imaging apparatus 100. The sectional view of the breast imaging apparatus 100 is a sectional view taken along a center line (indicated by alternate long and short dash line) extending in the vertical direction of the breast imaging apparatus 100 in FIG. 2.

As illustrated in FIG. 2, the front cover 26 for protecting the subject from the radiation generation unit 10 and the radiation detection unit 12 which rotate at CT imaging is provided on the gantry 30 on the CT imaging side. The front cover 26 has the opening 20 into which the breast of the subject to be CT imaged is inserted. Furthermore, the gantry 30 on the CT imaging side is provided with a plurality of holding portions 22a, 22b, 22c, and 22d to be held by the subject to be CT imaged. The plurality of holding portions 22a, 22b, 22c and 22d are each formed in a concave shape. The support leg portion 40 on the CT imaging side has a recess portion 48 to allow the subject to be CT imaged to enter a part of a subject's foot (feet).

As illustrated in FIG. 3, the pressing plate 14 for pressing the breast of the subject to be mammogram imaged is provided on the gantry 30 on the mammogram imaging side. A protection plate 4 for protecting the subject from an unnecessary radiation exposure is provided on the gantry 30 on the mammogram imaging side. The gantry 30 on the mammogram imaging side further includes a holding portion 70 to be held by the subject to be mammogram imaged. The holding portion 70 is formed in a projecting shape. The support leg portion 40 on the mammogram imaging side does not have the recess portion 48 to allow the subject to be mammogram imaged to enter the part of a subject's foot (feet).

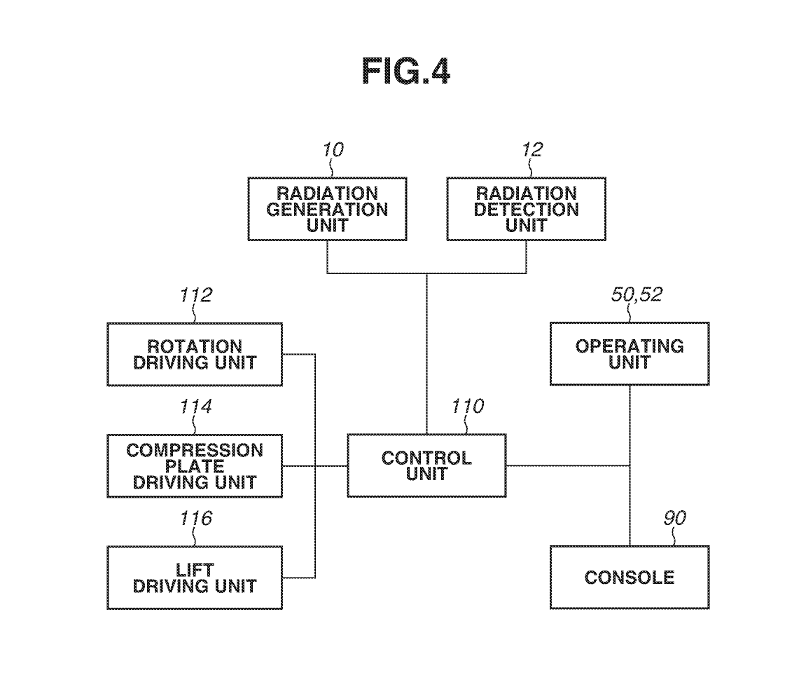

FIG. 4 illustrates a configuration diagram of the breast imaging apparatus 100. The breast imaging apparatus 100 includes a rotation driving unit 112 for rotating the radiation generation unit 10 and the radiation detection unit 12 in the state where the radiation generation unit 10 and the radiation detection unit 12 face each other. The breast imaging apparatus 100 further includes a pressing plate driving unit 114 for moving the pressing plate 14 up and down and a lift driving unit 116 for moving the gantry 30 up and down with respect to the support leg portion 40.

The breast imaging apparatus 100 includes a control unit 110 for controlling the radiation generation unit 10, the radiation detection unit 12, the rotation driving unit 112, the pressing plate driving unit 114, and the lift driving unit 116. Furthermore, the breast imaging apparatus 100 includes operating units 50 and 52 for transmitting an instruction to the control unit 110, and a console 90. The operating unit 50, with which the breast imaging apparatus 100 is operated, is provided on the gantry 30. The operating unit 52 that has the same function as the operating unit 50 is provided on a support base 2 that supports the radiation detection unit 12. In addition, the console 90 is provided outside of an imaging room.

Furthermore, the support base 2 may include a display unit for displaying at least one of subject information, height information of a radiation detection unit 12b, dose information of a radiation generation unit 10b, and pressing plate 14 pressure information (N) pressing plate.

The radiation generation unit 10 mainly includes an electron emission source that generates electrons and a target (not illustrated). The electrons generated by the electron emission source are emitted towards the target due to a potential difference between a cathode and an anode. The target is a member that generates the radiation as a result of collision of the electrons. The radiation generated by the target is emitted toward the outside, forming a cone beam shape. The control unit 110 can control a radiographing condition of the radiation generation unit 10.

The radiation detection unit 12 detects the radiation that has passed through the subject using a photoelectric transducer, and outputs the detected radiation as an electrical signal. For example, the radiation detection unit 12 includes a conversion panel for detecting radiation that has passed through the subject, an electricity storage unit, and an interface (I/F) outputting information converted into the electrical signal from the radiation. The interface (I/F) allows the electrical signal to be output to the control unit 110.

### <Gantry>

As illustrated in FIG. 2 and FIG. 3, the gantry 30 includes the ring-shaped rotation frame 38 for rotating the radiation generation unit 10 and the radiation detection unit 12 in the state where the radiation generation unit 10 and the radiation detection unit 12 face each other, and a ring-shaped stationary frame 30a for rotatably supporting the rotation frame 38. Furthermore, the stationary frame 30a is arc-like shape and may rotatably support a part of the rotation frame 38. The gantry 30 further includes a long cylinder unit 30b, which has a long cylinder shape, connected to the stationary frame 30a. The rotation frame 38 and the stationary frame 30a may be called a rotation unit for rotating the radiation generation unit 10 and the radiation detection unit 12. The stationary frame 30a and the long cylinder unit 30b are formed integrally. The stationary frame 30a is positioned higher than the long cylinder unit 30b. The long cylinder unit 30b is connected to the support leg portion. 40 for supporting the gantry 30 on the floor.

The gantry 30 stands in a vertical direction so that the subject can be radiographed in a standing posture. An axis of the rotation of the rotation unit (the rotation frame 38 in the gantry 30) for rotating the radiation generation unit 10 and the radiation detection unit 12 is a horizontal direction.

The long cylinder unit 30b covers an outer periphery of a long cylinder unit 42 in the support leg portion 40. More specifically, the long cylinder unit 42 in the support leg portion 40 is integrated inside of the long cylinder unit 30b in the gantry 30. The long cylinder unit 42 in the support leg portion 40 and the long cylinder unit 30b in the gantry 30 have a nested structure.

The breast imaging apparatus 100 includes the lift driving unit 116 for moving the long cylinder unit 30b up and down with respect to the support leg portion 40. In other words, the breast imaging apparatus 100 includes the lift driving unit 116 for moving the gantry 30 up and down.

### <Radiation Generation Unit and Radiation Detection Unit>

The breast imaging apparatus 100 includes the radiation generation unit 10 for generating the radiation and the radiation detection unit 12 for detecting the radiation emitted from the radiation generation unit 10. The radiation generation unit 10 and the radiation detection unit 12 are capable of rotating in the state where the radiation generation unit 10 and the radiation detection unit 12 face each other.

The radiation generation unit 10 and the radiation detection unit 12 are provided on the rotation frame 38 which rotates with respect to the stationary frame 30a of the gantry 30. Here, as illustrated in FIG. 3, the breast imaging apparatus 100 includes a radiation generation unit 10a and a radiation detection unit 12a for CT imaging, and the radiation generation unit 10b and the radiation detection unit 12b for mammogram imaging. The gantry 30 includes the radiation generation unit 10a and the radiation detection unit 12a for CT imaging, and the radiation generation unit 10b and the radiation detection unit 12b for mammogram imaging. In other words, the breast imaging apparatus 100 includes two pairs of the radiation generation unit and the radiation detection unit for CT imaging and mammogram imaging.

The gantry 30 includes the ring-shaped rotation frame 38 for rotating the radiation generation unit 10a and the radiation detection unit 12a for CT imaging, and the radiation generation unit 10b and the radiation detection unit 12b for mammogram imaging, in a state where the radiation generation unit 10a and the radiation detection unit 12a for CT imaging face each other, and the radiation generation unit 10b and the radiation detection unit 12b for mammogram imaging face each other.

More specifically, the radiation generation unit 10a and the radiation detection unit 12a for CT imaging are provided on the rotation frame 38. The radiation detection unit 12a is provided on the rotation frame 38 via the support base 2 for supporting the radiation detection unit 12a.

The radiation generation unit 10b and the radiation detection unit 12b for mammogram imaging are provided on the rotation frame 38. The radiation detection unit 12b is installed on the rotation frame 38 via the support base 2.

The rotation frame 38 is connected to the stationary frame 30a of the gantry 30 via a bearing which has a bearing structure. The stationary frame 30a is immobilized and a static frame. The rotation frame 38 is able to be rotated by the rotation driving unit 112. The rotation driving unit 112 is provided within the gantry 30 so that the axis of the rotation of the rotation frame 38 is the horizontal direction.

The pressing plate 14 is provided to the support base 2 to allow the pressing plate 14 to move up and down. Moreover, a rotation tab 54 for instructing the pressing plate 14 to move up and down is provided to the support base 2. By rotating the rotation tab 54 to lower the pressing plate 14, the breast of the subject is able to be pressed by the pressing plate 14 and the radiation detection unit 12b.

In this manner, the support base 2 is provided on the rotation frame 38. The support base 2 supports the radiation detection unit 12a, the radiation detection unit 12b, and the pressing plate 14. The radiation detection unit 12a and the radiation detection unit 12b are capable of rotating in a state where the rotate driving unit 112 rotates the rotation frame 38 with the support base 2. Moreover, the radiation generation unit 10a and the radiation generation unit 10b can be rotated in a state where the rotation driving unit 112 rotates the rotation frame 38.

As illustrated in FIG. 3, the radiation generation unit 10a and the radiation generation unit 10b are provided at substantially the same height. The radiation detection unit 12a is provided at a position higher than the radiation detection unit 12b.

In other words, the radiation generation unit 10a and the radiation generation unit 10b are provided so that the radiation generation unit 10a and the radiation generation unit 10b are at the same relative positions (i.e., with the same distance) from the axis of the rotation of a rotation unit (the rotation frame 38).

The radiation detection unit 12a and the radiation detection unit 12b are provided so that the radiation detection unit 12b is positioned outside of the radiation detection unit 12a with respect to the axis of the rotation of a rotation unit (the rotation frame 38).

A distance between the radiation generation unit 10a and the radiation detection unit 12a for CT imaging is shorter than a distance between the radiation generation unit 10b and the radiation detection unit 12b for mammogram imaging.

In mammogram imaging, the breast of the subject is pressed by the pressing plate 14 and the radiation detection unit 12b. Since the breast of the subject is pressed and become tabular, a field of view (FOV) should be secured by increasing an area to be irradiated with the radiation. Therefore, the radiation detection unit 12b for mammogram imaging is provided at a position lower than the radiation detection unit 12a for CT imaging.

A radiation field (i.e., FOV) 8 is a radiation field irradiated with the radiation from the radiation generation unit 10b for mammogram imaging. The radiation generation unit 10b and the radiation detection unit 12b are provided such that the radiation field 8 of the radiation generation unit 10b covers the pressing plate 14. The radiation field 8 is a quadrangular pyramid shape (cone beam shape) spreading from a focal point of the radiation generation unit 10b. As illustrated in FIG. 3, one side (left side in FIG. 3) of the radiation field 8 vertically extends. The other end (right side in FIG. 8) of the radiation field 8 obliquely extends. The radiation field 8 of the radiation generation unit 10b is determined so that a side (field end portion, field end surface) of the radiation field 8, the side (left side in FIG. 3) of the subject to be imaged by mammogram imaging, vertically extends, to radiograph an area around the breast (an axilla).

On the other hands, the radiation generation unit 10a and the radiation detection unit 12a are provided in such a manner that the size of the rotation frame 38 and the whole size of the breast imaging apparatus 100 (the gantry 30) become compact for CT imaging. Concretely, the radiation generation unit 10a and the radiation detection unit 12a are provided in such a manner that a distance between the radiation generation unit 10a and the radiation detection unit 12a is as short as possible. The radiation detection unit 12a is provided immediately below a breast holding unit 34. The radiation detection unit 12a is provided at a position that the radiation detection unit 12a is not brought into contact with the breast holding unit 34 even if the radiation detection unit 12a is rotated by the rotation frame 38.

A radiation field 6 is a radiation field irradiated with the radiation from the radiation generation unit 10a for CT imaging. The breast of the subject to be imaged by CT imaging is held on the breast holding unit 34 and is not pressed. The radiation generation unit 10a and the radiation detection unit 12a are installed in such a manner that the radiation field 6 of the radiation generation unit 10a covers a distal end portion of the breast holding unit 34.

The radiation field 6 is quadrangular pyramid shape (cone beam shape) spreading from a focal point of the radiation generation unit 10a. As illustrated in FIG. 3, one side (right side) of the radiation field 6 vertically extends. The other side (left side) of the radiation field 6 obliquely extends. The radiation field 6 is set in such a manner that a side (field end portion, field end surface) of the radiation field 6, the side (right side) of the subject to be imaged by CT imaging vertically extends, to radiograph the area around the breast (the axilla).

An described above, the breast imaging apparatus 100 according to the present exemplary embodiment includes the first radiation generation unit 10a for generating the radiation and the second radiation generation unit 10b for generating the radiation. The breast imaging apparatus 100 further includes the first radiation detection unit 12a for detecting the radiation emitted from the first radiation generation unit 10a and the second radiation detection unit 12b for detecting the radiation emitted from the second radiation generation unit 10b.

The breast imaging apparatus 100 performs radiograph using the first radiation generation unit 10a and the first radiation detection unit 12a in the state where an imaging target portion of the subject on the first side of the breast imaging apparatus 100 is pressed between the pressing plate 14 and the first radiation detection unit 12a. Furthermore, the breast imaging apparatus 100 performs the radiograph while rotating the second radiation generation unit 10b and the second radiation detection unit 12b in the state where the imaging target portion of the subject is inserted between the second radiation generation unit 10b and the second radiation detection unit 12b on a second side opposite to the first side of the breast imaging apparatus 100.

In this manner, the breast imaging apparatus 100 includes two pairs of the radiation generation unit and the radiation detection unit for CT imaging and mammogram imaging. Therefore, it is possible to obtain FOVs suitable for the breast of the subject for CT imaging and the breast of the subject for mammogram imaging.

### <Support Leg Portion (Recess Portion)>

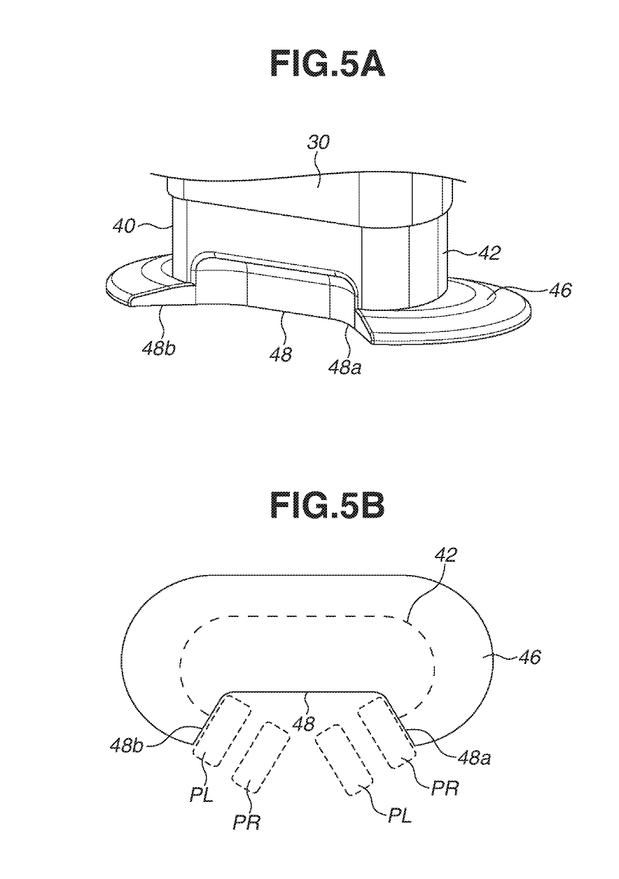

FIG. 5A and FIG. 5B each illustrate the support leg portion 40 of the breast imaging apparatus 100. As illustrated in FIG. 2, FIG. 3, FIG. 5A, and FIG. 5B, the breast imaging apparatus 100 includes the gantry 30 for rotatably supporting the imaging unit 102 and the support leg portion 40 for supporting the breast imaging apparatus 100 (the gantry 30) on the floor in the vertical direction. The support leg portion 40 supports the breast imaging apparatus 100 (the gantry 30) in the vertical direction.

The support leg portion 40 includes the long cylinder portion 42 which is formed by being extended in a direction (a vertical direction) that is orthogonal to a horizontal plane and a base unit 46 which is connected to the long cylinder portion 42 and supports the breast imaging apparatus 100 (the gantry 30) stably. The base unit 46 is a member which is in contact with the floor. The long cylinder portion 42 of the support leg portion 40 is integrated with the base unit 46 of the support leg portion 40. The long cylinder portion 42 is connected to the gantry 30.

The base unit 46 is in contact with the floor. The base unit 46 is formed in accordance with a peripheral shape of the long cylinder portion 42. The long cylinder portion 42 has a cylinder form of an ellipse shape with two semicircles being connected via straight lines. The base unit 46 is formed in an ellipse shape, because the long cylinder portion 42 has a cylinder form of the ellipse shape. The long cylinder portion 42 and the base unit 46 are in a similarity relation.

Concretely, the base unit 46 is formed in such a manner that the base unit 46 protrudes (projected) outward from the circumference of a bottom face of the long cylinder portion 42. In other words, the base unit 46 is formed in such a manner that the base unit 46 uniformly protrudes outward from the circumference of the bottom face of the long cylinder portion 42. The base unit 46 is formed in such a manner that the base unit 46 uniformly protrudes outward from the circumference of a bottom face of the long cylinder portion 42 by a constant-length (for example, around 10 cm-30 cm).

A contact area where the base unit 46 contacts the floor is wider than an area of the bottom face of the long cylinder portion 42. By expanding the area of the bottom face of the long cylinder portion 42 with the base unit 46, the breast imaging apparatus 100 can have an increased contact area with the floor. An installation balance of the breast imaging apparatus 100 (the gantry 30) can be maintained with the base unit 46. The breast imaging apparatus 100 (the gantry 30) can be supported stably.

As illustrated in FIG. 5A and FIG. 5B, the support leg portion 40 for supporting the breast imaging apparatus 100 on the floor includes the recess portion 48 to allot the subject to enter a part of the subject's foot (feet). The recess portion 48 has a concave shape. FIG. 5A is a diagram illustrating the recess portion 48 of the support leg portion 40. FIG. 5B is a diagram illustrating a bottom face of the base unit 46 and a position at which the feet of the subject are positioned.

As illustrated in FIG. 5B, a distal end portion (a toe) of a foot of the subject is inserted into the recess portion 48. The recess portion 48 is opened to an extent that the distal end portions (toes) of feet of the subject can be inserted. More specifically, the recess portion 48 is formed in such a manner that a part of the base unit 46 on a predetermined side is cut. In other words, the recess portion 48 is formed in such a manner that a hole is formed inside of the base unit 46 on the predetermined side. For example, the depth of the recess portion 48 is about 20 cm; the height of the recess portion 48 is about 10 cm from the floor. Furthermore, the recess portion 48 may be formed such that a part of the long cylinder portion 42 on the predetermined side is cut off with the base unit 46. The predetermined side is the CT imaging side where the subject is standing in CT imaging. The CT imaging side means the front side of the breast imaging apparatus 100 in FIG. 2 and the right side of the breast imaging apparatus 100 in FIG. 3.

As illustrated in FIG. 3, the breast imaging apparatus 100 performs CT imaging while rotating the radiation generation unit 10 and the radiation detection unit 12 with the rotation frame 38 in a state where the breast of the subject is inserted into an opening 20 of the front cover 26. In CT imaging, the subject faces the breast imaging apparatus 100 side and an upper part of the subject tightly contacts the front cover 26.

When a perpendicular line is brought down from an end surface of the front cover 26, the perpendicular line arrives at the base unit 46. Therefore, if there is not the recess portion 48 in the base unit 46, the feet of the subject must be put on the base unit 46. In that case, CT imaging might be performed in a state where the posture of the subject is not stable.

Accordingly, in the breast imaging apparatus 100 according to the present exemplary embodiment, the base unit 46 has the recess portion 48 to allow the subject to enter a part of the subject's foot (feet). The recess portion 48 is formed so that an area of the floor corresponding to the recess portion 48 is not covered by the base unit 46. Therefore, the subject inserts the feet into the recess portion 48 and puts the feet on the floor. After that, the subject can keep a posture in a state where the subject faces the breast imaging apparatus 100 side. CT imaging can be performed in a state where the posture of the subject is stable.

In addition, in consideration of an installation balance of the breast imaging apparatus 100 (the gantry 30), a part of the base unit 46 on the opposite side to the predetermined side is not cut off. In other words, the recess portion 48 is formed in such a manner that the breast imaging apparatus 100 (the gantry 30) does not lose the installation balance.

The base unit 46 protrudes on both the CT imaging side and the mammogram imaging side in a similar way. The base unit 46 has the protruded part, and the base unit 46 on CT imaging side has the recess portion 48 to allow the subject to enter a part of the subject's foot (feet).

As illustrated in FIG. 5B, the long cylinder portion 42 and the base unit 46 is an ellipse shape with two semicircles connected via straight lines, and a part of the ellipse shape is cut off, if the breast imaging apparatus 100 is viewed from a top of the breast imaging apparatus 100. The recess portion 48 is formed in such a manner that a straight line part on the CT imaging side is cut off from the long cylinder portion 42 of the ellipse shape. Furthermore, the recess portion 48 is formed in such a manner that a straight line part on CT imaging side is cut off from the base unit 46 of the ellipse shape. Moreover, the semicircles of the base unit 46 are not cut off. More specifically, the recess portion 48 is formed in such a manner that a circumference portion (an edge portion) of the base unit 46 necessary for the installation balance of the breast imaging apparatus 100 (the gantry 30) remains (the circumference portion (the edge portion) of the base unit 46 can be secured).

As illustrated in FIG. 5B, the recess portion 48 is formed by expanding an opening from a back end face of the recess portion 48. The recess portion 48 is formed to become the linear symmetry (bilateral symmetry). The recess portion 48 is formed to become a substantially trapezoidal shape with both an inclined surface 48a and an inclined surface 48b, if the breast imaging apparatus 100 is viewed from top of the breast imaging apparatus 100. Each of the inclined surface 48a and the inclined surface 48b are slanted to the back end face of the recess portion 48. The inclined surface 48a and the inclined surface 48b are orthogonal to the floor.

When the breast imaging apparatus 100 implements CT imaging of a right breast of the subject, a right foot PR of the subject is set to contact the inclined surface 48a of the recess portion 48. The subject can keep diagonally forward right posture to the front cover 26, in other words, the subject can obliquely face the front cover 26 leftward, so that the subject can set the right breast in the front cover 26 close compared with the left breast. Therefore, it is easy to insert the right breast to the opening 20 of the front cover 26, and the breast imaging apparatus 100 can stably implement CT imaging without the subject losing the posture.

In a similar way, when the breast imaging apparatus 100 implements CT imaging of a left breast of the subject, a left foot PL of the subject is set to contact the inclined surface 48b of the recess portion 48. The subject can keep diagonally forward left posture to the front cover, in other words, the subject can obliquely face the front cover 26 rightward, so that the subject can set the left breast in the front cover 26 close compared with the right breast. Therefore, it is easy to insert the left breast to the opening 20 of the front cover 26, and the breast imaging apparatus 100 can stably implement CT imaging without losing the posture.

As described above, the base unit 46 on the CT imaging side has the recess portion 48 to allow the subject to enter a part of the subject's foot (feet). On the other hand, as illustrated in FIG. 2 and FIG. 3, the base unit 46 on the mammogram imaging side, on which the subject is positioned in mammogram imaging, does not include the recess portion 48 to allow the subject in mammogram imaging to enter a part of the subject's foot (feet). In other words, the support leg portion 40 on the mammogram imaging side does not include the recess portion 48. In fact, the support leg portion 40 has the recess portion 48 only on the CT imaging side (right side in FIG. 3). This is because composition elements such as the radiation generation unit 10b, the radiation detection unit 12b, and the pressing plate 14 protrude to the mammogram imaging side (left side in FIG. 3), i.e., the subject side (left side in FIG. 3) where the breast imaging apparatus 100 implements mammogram imaging. Composition elements such as the radiation generation unit 10b, the radiation detection unit 12b, and the pressing plate 14 protrude toward the subject side (left side) where the breast imaging apparatus 100 implements mammogram imaging further than the base unit 46. Therefore, the recess portion 48 is not necessary for the base unit 46 on the mammogram imaging side.

As described above, the breast imaging apparatus 100 according to the present exemplary embodiment includes the support leg portion. 40 for supporting the gantry 30 on the floor. The support leg portion 40 includes a recess portion 48 to allow the subject to enter a part of the subject's foot. (feet).

The breast imaging apparatus 100 performs the radiograph while rotating the radiation generation unit 10 and the radiation detection unit 12 in the state where an imaging portion to be imaged of the subject is inserted between the radiation generation unit 10 and the radiation detection unit 12. The support leg portion 40 on the CT imaging side where the subject is standing in the radiograph imaging includes the recess portion 48 to allow the subject to enter a part of the subject's foot (feet).

Furthermore, a recess portion in which the feet of the subject are entered is not provided to the support leg portion 40 on the mammogram imaging side, where the subject is positioned when the breast imaging apparatus 100 performs the radiograph using the radiation generation unit 10 and the radiation detection unit 12 in the state where the breast of the subject is pressed between the pressing plate 14 and the radiation detection unit 12. Therefore, the subject can keep a suitable posture for each imaging. The breast imaging apparatus 100 can perform CT imaging in the state to fix the breast of the object properly.

### <Holding Unit>

As illustrated in FIG. 2, the gantry 30 on the CT imaging side includes a plurality of holding portions 22a, 22b, 22c and 22d for the subject to be CT imaged to hold. Concretely, each of the plurality of holding portions 22a, 22b, 22c and 22d is formed along circumferential direction of the circumference of the ring-shaped stationary frame 30a, which rotatably supports the rotation frame 38 of the gantry 30. Furthermore, the plurality of holding portions 22a, 22b, 22c and 22d are formed near a bonding portion between the stationary frame 30a and the front cover 26 provided on the stationary frame 30a.

The holding portion 22a is formed at an upper right portion, the holding portion 22b is formed at a lower right portion, the holding portion 22c is formed at an upper left portion, and the holding portion 22d is formed at a lower left portion on the stationary frame 30a. The holding portions on the stationary frame 30a may be formed right above the subject (not illustrated).

The plurality of holding portions 22a, 22b, 22c and 22d are concave shaped holding portions. The plurality of holding portions 22a, 22b, 22c and 22d are formed on the ring-shaped stationary frame 30a. The holding portions 22 are concaved to an extent that a finger-tip of the subject is caught on.

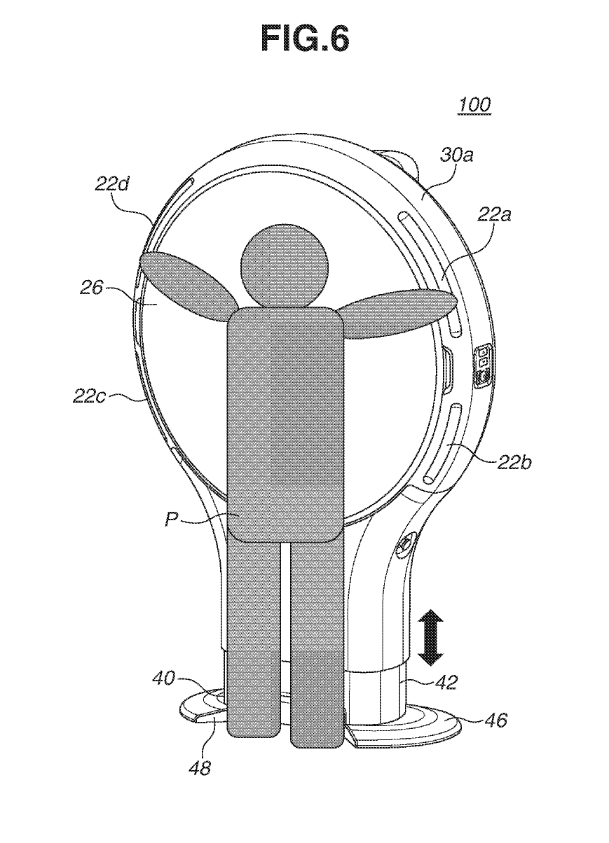

FIG. 6 is a diagram illustrating an image-capturing state of a subject P during CT imaging. The subject P holds at least one of the plurality of holding portions 22a, 22b, 22c and 22d. As illustrated in FIG. 6, the subject P can hold the holding portion 22a and the holding portion 22d on the upper side of gantry 30. At this time, the distal end portion (the toe) of the foot of the subject P is inserted into the recess portion 48. The subject P can support its body by bringing the body of the subject P to the side of gantry 30.

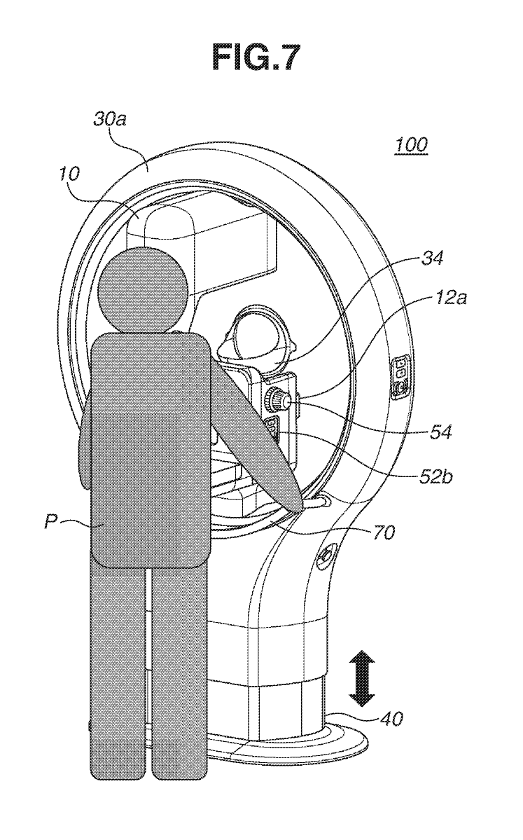

FIG. 7 is a diagram illustrating an image-capturing state of the subject P during mammogram imaging. The subject P holds a holding portion 70 with both hands. The subject P can support its body by bringing the body of the subject P to the side of the gantry 30.

The distance between both support points of the holding portion 70 is longer than a width of the radiation generation unit 10b or the radiation detection unit 12b. Therefore, the holding portion 70 is not hidden by the radiation generation unit 10b and the radiation detection unit 12b, even if the radiation generation unit 10b and the radiation detection unit 12b are rotated. Accordingly, the subject can hold the holding portion 70, even if the radiation generation unit 10b and the radiation detection unit 12b are rotated.

The plurality of holding portions 22a, 22b, 22c and 22d on the CT imaging side is different from the holding portion 70 on the mammogram imaging side in terms of their shape. The holding portions 22a, 22b, 22c, and 22d on the CT imaging side are recessed with respect to the gantry 30. On the other hand, the holding portion 70 on the mammogram imaging side projects with respect to the gantry 30.

On the CT imaging side, it is necessary that an upper part of the subject body tightly contacts the front cover 26 and that the breast of the subject, imaging targeted portion, is inserted into the opening 20. For that reason, holding portions 22a, 22b, 22c and 22d on the CT imaging side do not project beyond the gantry 30 not to obstruct the subject that is stuck to the front cover 26. Each of holding portions 22a, 22b, 22c, and 22d on CT imaging side is formed in a concave shape.

On the mammogram imaging side, the upper part of the subject body does not firmly contact the front cover 26. It is necessary that the breast of the subject is inserted and pressed between the pressing plate 14 projecting from the gantry 30 and the radiation detection unit 12. Moreover, it is necessary that the body of the subject is supported in the state where the breast of the subject is pressed. Accordingly, the holding portion 70 on the mammogram imaging side projects beyond the gantry 30. The holding portion 70 on the mammogram imaging side is formed in a projecting shape.

As described above, the gantry 30 on the first side of the breast imaging apparatus 100 according to the present exemplary embodiment includes a first holding portion 70 for the subject to hold. The gantry 30 on the second side opposite to the first side includes a second holding portion 22 that is different from the first holding portion 70 in terms of their shape. The first holding portion 70 is formed in a projecting shape with respect to the gantry 30. The second holding portion 22 is formed in a concave shape with respect to the gantry 30.

Therefore, the subject for mammogram imaging and the subject for CT imaging hold the first holding portion 70 and the second holding portion 22 properly. As a result, the subject can keep a suitable posture for each imaging.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2015-132186, filed Jun. 30, 2015, which is hereby incorporated by reference herein in its entirety.

What is claimed is:

1. A breast imaging apparatus comprising:
a gantry including a radiation generation unit configured to generate a radiation and a radiation detection unit configured to detect the radiation emitted from the radiation generation unit; and
a support leg portion configured to support the gantry with respect to a floor,
wherein the radiation generation unit and the radiation detection unit are capable of rotating in a state where the radiation generation unit and the radiation detection unit face each other, and
wherein the support leg portion has a recess portion configured to allow a part of a foot (feet) of a subject to enter therein.

2. The breast imaging apparatus according to claim 1, wherein the support leg portion is formed with a long cylinder portion extending in a vertical direction and a base unit which is connected to the long cylinder portion and in contact with the floor.

3. The breast imaging apparatus according to claim 2, wherein the base unit is formed in such a manner that the base unit protrudes outward from a circumference of a bottom face of the long cylinder portion.

4. The breast imaging apparatus according to claim 2, wherein the recess portion is formed such that a part of the base unit on a predetermined side is cut off.

5. The breast imaging apparatus according to claim 2, wherein the recess portion is formed such that a part of the long cylinder portion on the predetermined side is cut off with the part of the base unit.

6. The breast imaging apparatus according to claim 4, wherein the recess portion is formed so that the floor is not covered in an area of the base unit corresponding to the recess portion.

7. The breast imaging apparatus according to claim 4, wherein the predetermined side is a CT imaging side where the subject is positioned in CT imaging.

8. The breast imaging apparatus according to claim 7, wherein the support leg portion on a mammogram imaging side where the subject is positioned in mammogram imaging does not include the recess portion.

9. The breast imaging apparatus according to claim 1, wherein the recess portion is formed by expanding an opening from a back end face of the recess portion.

10. A breast imaging apparatus comprising:
a gantry including a radiation generation unit configured to generate a radiation and a radiation detection unit configured to detect the radiation emitted from the radiation generation unit; and
a support leg portion configured to support the gantry with respect to a floor,
wherein the radiation generation unit and the radiation detection unit are capable of rotating in a state where the radiation generation unit and the radiation detection unit face each other, and
wherein the support leg portion of a CT imaging side includes a recess portion configured to allow a part of a foot (feet) of a subject to enter therein, the CT imaging side on which the subject is positioned in performing radiograph while the radiation generation unit and the radiation detection unit are rotated in a state where the breast of the subject is inserted between the radiation generation unit and the radiation detection unit.

11. The breast imaging apparatus according to claim 10, wherein the support leg portion on a mammogram imaging side does not include the recess portion configured to allow a part of a foot (feet) of the subject to enter therein, the mammogram imaging side on which the subject is positioned in performing the radiograph using the radiation generation unit and the radiation detection unit in a state where the breast of the subject is pressed between a pressing plate and the radiation detection unit.
